(19) United States
(12) Patent Application Publication
(10) Pub. No.: US 2019/0388062 A1
(43) Pub. Date: Dec. 26, 2019

(54) Apparatus, System, and Method for Increasing Object Visibility

(71) Applicant: Microtech Medical Technologies, Ltd., Tel Aviv (IL)

(72) Inventor: Amir KOHEN, Tel Aviv (IL)

(21) Appl. No.: 16/445,383

(22) Filed: Jun. 19, 2019

Related U.S. Application Data
(60) Provisional application No. 62/687,398, filed on Jun. 20, 2018.

(51) Int. Cl. A61B 8/08; A61B 10/02; A61F 2/24; A61L 31/14; A61L 31/02; A61L 31/06; A61B 17/34

(52) U.S. Cl. CPC A61B 8/481; A61B 10/0233; A61B 8/488; A61B 8/0841; A61B 2010/045; A61L 31/14; A61L 31/022; A61L 31/06; A61B 17/3403; A61F 2/2427

(57) ABSTRACT

The invention provides an apparatus, system, and method employing a marker based on a membrane that is reflective of ultrasonic waves. The technique allows controlling the resonance frequency of the membrane, amplitude, non-linearity and reverberation period by modifying material properties and geometrical properties (membrane area and thickness) such that the marker appears prominently on ultrasound imaging. The marker can be tailored for use either in color Doppler mode or harmonics mode.

This application claims the benefit of priority of U.S. Provisional Application Ser. No. 62/687,398 filed Jun. 20, 2018, which is incorporated by reference in its entirety.

### FIELD OF INVENTION

The invention relates to an apparatus, system, and method for increasing object visibility using ultrasound imagery. In particular, the apparatus includes novel features to facilitate a distinctive visual fingerprint using ultrasound.

### BACKGROUND OF THE INVENTION

Standard ultrasound imagery is based on object reflectance due to acoustic impedance mismatch. Ultrasound machines also have the ability to detect and superimpose on the image the changes in frequency of the reflected wave with respect to the transmitted one. Ultrasound machines are able to work in a variety of methods. Doppler imagery is commonly used to detect blood flow which causes a shift in the ultrasound frequency of the value shown in the equation

$$Δ f ≅ f ( 1 + \frac{v cos ( θ )}{c} ) ,$$

where f is the ultrasound probe frequency, c is the speed of sound, v is the blood velocity, and θ is the angle between the ultrasound probe and the blood velocity. Commonly-used ultrasound imagers are capable of detecting a Δf on the order of one to ten kHz. In harmonic imagery, the ultrasound is reflected by a tissue with non-linear properties resulting in reflectance of frequencies given by fᵣ=n*f where n is an integer.

In general, an ultrasound imager translates the reflected signal to an image depending on the amplitude of the reflected wave, its time in flight and its frequency. The latter is sometimes affected by reverberations which the imager interprets as signals coming from a longer distance.

Currently, ultrasound imaging is used to provide a noninvasive method of creating a visual image or signal of what constitutes an opaque or mostly opaque membrane, which can be used in many different circumstances, for example, diagnostic tests evaluating different issues ranging, for example, from fetal health to artery deterioration. Further, ultrasound imaging is often used to guide needles being used for many different medical purposes including biopsies.

Currently available methods of visualizing implanted or invasive devices using ultrasound cannot distinguish such devices from body parts. Difficulty in differentiating implanted devices, invasive devices, and/or needles, for example, means that more invasive procedures must be used in examinations and procedures for device detection. Further, difficulty in identifying needles, for example, using ultrasound guidance results in practitioners struggling to guide these devices during surgical procedures. This results in biopsies and other procedures taking longer than necessary and causing patients unnecessary discomfort due to guidance mishaps and misplacement of needles

U.S. Pat. No. 9,138,286 (assigned to NuVue Therapeutics Inc.) describes an interventional medical device for use with a motion-sensitive ultrasonic imaging system which is intended to determine the location of diagnostic or therapeutic probes in soft tissue. A two-axis flexing mechanism in the medical device induces an orbital movement of an elongate member of the device to vibrate. According to the patent, this motion causes local agitation of fluids and/or tissues surrounding the elongate member and the agitation is visible on an ultrasound system sensitive to motion.

NuVue Therapeutics has FDA marketing approval for a product called “NuVue ColorMark Needle”, which is intended to allow the visualization by ultrasound of a needle, such as a biopsy needle, inserted into the body. According to NuVue, the product is a hand-held needle device consisting of a battery-operated single-use device that integrates and vibrates interventional biopsy needles. The operating principle of the device is that the needle is made to vibrate at low sonic frequencies which make it visible on a Doppler Color-flow system since the vibration of the needle appears as motion to the Doppler system.

Some visualization systems exist for viewing ultrasound data. For example, RealView Imaging (Yokneam, Israel) provides a system for imaging of 3D structures generated from medical 3D volumetric data using interference-based holography.

Although these visualization methods have provided useful advances in the field. they have not solved the problem of identifying the real location of surgical, diagnostic, or therapeutic medical objects such as biopsy needles, probes, and sensors in the body via ultrasound during an invasive procedure or after implantation.

As such, a need exists to make invasive and implanted devices more easily and particularly visible using ultrasound imaging. Moreover, a broader need exists to detect the location of the medical device in a body cavity.

### SUMMARY OF THE INVENTION

The invention relates to medical devices having a marker which can be detected with ultrasound imaging wherein the marker comprises a vibrating membrane, reflective of ultrasonic waves. The marker may further comprise a housing wherein the membrane is attached to the housing at its edges. The vibrating membrane has an area and a thickness such that the membrane vibrates at a specific resonance frequency, amplitude, non-linearity, and reverberation period in response to a vibrating stimulation. Three preferred types of ultrasound detection are preferred, that is, color Doppler imagery, acoustic radiation force impulse (or “ARFI”) imaging, and harmonic imagery. Other ultrasound methods may also be utilized.

In certain embodiments, the marker comprises a housing and a vibrating membrane which may be an air-backed membrane. The membrane may be made of one or more suitable biocompatible materials such as silicon, titanium, or other similar biocompatible materials. The housing may also be made of one or more suitable biocompatible materials such as silicon, titanium, or other similar biocompatible materials.

The invention relates to a detectable invasive medical device system comprising an invasive medical device and a marker having a membrane reflective to ultrasonic waves. The marker may be attached to any invasive medical device, that is, a device which is either implanted in a permanent or temporary manner, or a medical device inserted into the body. For example, an invasive medical device may include a needle, a biopsy needle, catheter, angioplasty catheter, or other invasive or surgical device. Alternatively, the marker may be attached to a temporary or permanent implant such as, for example, a stent, heart valve, defibrillator, sensor, or other implant. The marker may be attached thereto via welding, adhesive, or other similar means or integrated into the device during manufacture.

Detection of the marker utilizes ultrasound techniques. In one embodiment, the marker may have a resonance frequency equal to that of the ultrasound imager of a harmonics-type ultrasound imaging. In another embodiment, the marker may have a resonance frequency in the range accessible to color Doppler imagers. In yet other embodiments, the marker may have a resonance frequency larger than that of the Doppler shift created by blood flow. In yet further embodiments, the membrane has a resonance frequency accessible to ARFI imagers. Some of these techniques may use a first and a second transducer, in which the first transducer is used to excite the marker and the second transducer is used to transmit images. Some techniques will use one transducer for both excitation and image transmission.

The present invention also relates to a system comprising a marker device having a membrane and a housing, wherein the membrane is reflective of ultrasonic waves, and an ultrasound generating device. In this system, the membrane has an area and a thickness which allows the membrane to vibrate at a specific resonance frequency, amplitude, non-linearity, and reverberation period. The system includes an ultrasound generating device such as an ultrasound transducer. The ultrasound transducer preferably transmits at a frequency range corresponding to the resonance frequency of the membrane.

The invention also relates to a method for using the system. The method includes the steps of (1) positioning the medical device having a marker membrane at a target position in a body cavity, (2) placing an ultrasound transducer on or near an outer surface of said target position to excite the marker membrane, and (3) detecting said excited marker vibration via a detectible signal indicating the location of the medical device. The method may use any of the embodiments of the marker or system described herein.

### BRIEF DESCRIPTION OF THE DRAWINGS

FIG. 1A shows detection of true and false catheter tips using ultrasound.

FIG. 1B shows an ultrasound image of a possible false tip.

FIG. 2A shows a side view of an exemplary air-backed membrane of the invention.

FIG. 2B shows a front view of an exemplary air-backed membrane of the invention.

FIG. 3 shows a needle incorporating an exemplary ultrasound marker.

FIG. 4 shows a pacemaker incorporating an exemplary ultrasound marker.

FIG. 5A shows an isometric view of an exemplary ultrasound marker of the invention.

FIG. 5B shows a side view of an exemplary ultrasound marker of the invention.

FIG. 5C shows a top view of an exemplary ultrasound marker of the invention.

### DETAILED DESCRIPTION OF THE INVENTION

The invention relates to a medical device having an ultrasound marker. The marker comprises a membrane reflective of ultrasound waves. The invention also relates to systems and methods for using the ultrasound marker in detecting an invasive medical device.

The ultrasound marker mounted on or integrated into the medical device is designed to have a specific resonance frequency which appears distinct in an ultrasound image. The resonance frequency can be determined by the geometric properties of the marker. The marker is preferably less than 0.50 mm³ in size, having any suitable shape and dimension. One example marker is the sensor device described in U.S. Pat. No. 6,770,032 to Kaplan which is incorporated herein in toto by reference. This marker may be similar to a sensor device comprising surfaces of 10-2000 micrometers in each dimension. In other embodiments, the marker is not dependent on pressure. Because the Kaplan sensor must be sensitive to pressure, it is required to have a ratio of thickness to diameter of approximately 1:100. The marker of the present invention does not require sensitivity to pressure. The membrane is most effective when the ratio of thickness to diameter is between 1:4 and 1:6, but in certain embodiments, the ratio of thickness to diameter is less than 1:100, and may be as low as 1:3, for example.

In the prior art, such as U.S. Pat. No. 6,770,032 to Kaplan, the housing which protects the marker, transfers the pressure and is largely transparent to ultrasound. In the present invention, there is no need to transfer the pressure from outside the housing to within the housing. The housing's main purpose is to protect the marker from deterioration which can occur within the body. Another example marker is the sensor device described in U.S. Pat. No. 7,134,341 to Girmonsky. Moreover, prior art sensors require short broadband ultrasonic pulses. The invention may use marker devices having longer broadband ultrasonic pulses. Such pulses can range from microseconds to continuous waves.

In one embodiment, the marker is incorporated into an invasive medical device by mounting said marker thereon or integrating into or onto the medical device. A body cavity includes any internal body space, organ, or lumen which is not visible from outside the body. The marker may be integrated into the medical device by being attached in one of a number of ways including welding, embedding or integrating during manufacture. Some devices will require markers be made of specific materials to prevent corrosion or to assist in bonding. An invasive medical device includes those used in surgical procedures or permanent or temporary implants including, for example, artificial joints, artificial hips, artificial knees, heart pacemakers, breast implants, intra-uterine devices, implanted screws, implanted pins, implanted plates, implanted rods, spine screws, pine rods, artificial spine disks, coronary stents, ear tubes, and vascular access ports. More generically, the invasive medical device is one used or placed inside a body cavity.

The invention also relates to a method of detecting the invasive medical device using the ultrasonic marker. The method may comprise the steps of (a) placing an invasive medical device having a marker within a body cavity, including an artery, ear cavity, mouth, or injection site, for example; (b) directing ultrasonic waves from an outer surface of the body cavity toward the medical device using an ultrasonic transducer, causing the marker to vibrate at its resonance frequency; and (c) detecting the medical device and its location in the body cavity. The method of the invention may detect an implanted device and may include the steps of: (1) directing ultrasound waves from an outer surface of the body toward the implanted device having the marker; and (2) detecting signals from the marker thereby determining the location of the implanted device.

Ultrasonic transducers convert standard AC electrical current into ultrasound waves, as well as the reverse. Ultrasonic transducers typically refer to piezoelectric transducers or capacitive transducers. Piezoelectric crystals may be used in that such crystals change size and shape when a voltage is applied. Specifically, AC voltage makes the piezoelectric crystals oscillate at a consistent frequency and produce ultrasonic sound. Because piezoelectric materials generate a voltage when force is applied to such materials, piezoelectric devices can also work as ultrasonic detectors. Some ultrasonic systems may use separate transmitters and receivers, while others may combine both functions into a single piezoelectric transceiver. Alternatively, capacitive transducers may use electrostatic fields between a conductive diaphragm and a backing plate to generate ultrasound waves. Both types of transducers are known in the art and may be used in the invention.

Different transducers are needed for different ultrasound imaging systems. The marker of the invention may, for example, be detected using color Doppler ultrasound, continuous wave Doppler imaging, ARFI imaging, or harmonic imaging ultrasound.

Color Doppler imaging is a preferred ultrasonic technique for medical imaging because such imaging permits visualization of moving objects relative to a static background. Use of color Doppler imaging may require a physician to be able to aim the detection device accurately relative to the target. Measurement of Doppler shifts permits separation of moving objects from the background, such as movement of blood cells or the beating of the heart (as in an echocardiogram). Often, the moving objects are assigned a color depending on the direction of their motion and the static background is depicted in grayscale. The marker of the present invention may be displayed as a distinct color in the image.

A color Doppler-type ultrasonic diagnostic apparatus functions by detecting frequency shifts, caused by the Doppler effect of ultrasonic pulses transmitted toward an object being examined by means of frequency analysis of received echo signals, and displaying a distribution image of a flow of blood based on the detected results. The detectable frequency shift (hereinafter, referred to as a Doppler shift component) corresponds to a component of velocity in an ultrasonic beam direction.

In another embodiment, the marker may be detected using harmonic imaging. In conventional ultrasound imaging, the ultrasound system transmits and receives a sound pulse of a specific frequency. The returned signal in traditional ultrasound imaging is less intense than the transmitted signal, losing strength as the signal passes through tissue. In harmonic imaging, the signal returned by the target includes not only the transmitted fundamental frequency, but also signals of other frequencies—most notably, the harmonic frequency, which is twice the fundamental frequency. Once this combined fundamental/harmonic signal is received, the ultrasound system separates out the two components and then processes the harmonic signal alone.

Harmonics are generated by the passage of the ultrasound scanner beam as the harmonic passes through tissue. The peaks and troughs of the transmitted pulse cause the target to alternatively expand and contract, distorting its shape. Because a target tissue, for example, is not linearly elastic, the tissue contracts less than it expands. During tissue contraction, tissue density increases, causing the peak of the sound wave to travel slightly faster than the trough. The result of this process, called non-linear propagation, is that the wave becomes progressively more asymmetrical. This asymmetrical distortion results in harmonics.

Although the amount of harmonics that a target tissue generates at any given instant is small, the harmonics build as the pulse propagates through the tissue. Thus, as the ultrasound scanner wave travels through more tissue, more harmonics are generated. In an environment in which tissue appears so prominently on an ultrasound image, detecting traditional medical devices, implants, needles, and catheters without a marker surrounded by tissue is difficult and imprecise. The invention comprises a medical device having a marker which appears more prominently than tissue allowing easy detection.

Higher intensity transmission waves generate both higher intensity harmonics and more harmonics. The production of the second harmonic is proportional to the square of the fundamental intensity. Thus, a 3-dB increase in the fundamental beam will result in a 6-dB increase in harmonic intensity. For this reason, harmonics are generated predominantly by the main transmit beam. The region of maximal production of harmonics is at the focal zone because beam intensity is highest at that location. Little or no harmonics are produced by weak waves, such as side lobes, grating lobes, scattered echoes, and at the edges of the main ultrasound scanner beam. As a result, beams formed from harmonic signals have less noise, and improved contrast resolution. The resonance frequency of the marker of the invention may be designed to be the frequency of the transducer, which will cause it to appear more intensely and brighter on the ultrasound image.

Tissue harmonic imaging using ultrasound scanners operate by transmitting a fundamental beam having a lower frequency. The resulting fundamental pulse propagates through tissue inside the body, and generates the higher frequency harmonic sound. Tissue harmonic imaging in ultrasound scanners forms the image primarily from the higher frequency harmonic sound. Echoes from the fundamental frequency are rejected and thus, are not used in generating the image. Indeed, if the higher amplitude fundamental echoes are not eliminated, the echoes degrade the image to the point that there is no benefit from tissue harmonic imaging. Further, the echoes cause much stronger signal, but are not relevant to the medical device. Advanced transmit beam formation and signal detection is preferred to produce good quality harmonic images.

In yet another embodiment of the invention, the medical device may be detected using ARFI imaging. In acoustic radiation force impulse (ARFI) imaging, a single ultrasound transducer is used to both induce and monitor on-axis deformation in order to generate qualitative images of tissue stiffness. At a single lateral location, an ARFI pulse sequence consists of three pulse types: 1) reference pulses that precede the ARF burst and may be used as a baseline for tissue position, 2) pushing pulses that consist of a high-intensity impulse to displace the tissue, and 3) tracking pulses that monitor the tissue dynamics following the ARF burst. Tissue displacement is measured using correlation or phase-shift estimation techniques on raw RF data from the reference and tracking pulses. The pulse sequence and motion estimation is repeated at a number of lateral positions across the imaging field of view to build up a 3D data set consisting of tissue displacement as a function of axial position, lateral position and time.

The membrane used as the ultrasonic marker may be an air-backed membrane. The air-backed membrane prevents acoustic leakage compared to other membranes. Specifically, the air-backed membrane used in the present invention reduces or eliminates energy dissipation and reflects the ultrasound waves. The air-backed membrane is suspended on a frame to vibrate. For example, the housing may form a geometric solid, and the air-backed membrane may be a cylinder attached thereto. In one embodiment, a circular air-backed membrane may, for example, have the dimensions of approximately 0.3 to 2.0 mm diameter×0.05 to 0.50 mm thickness. In exemplary embodiments, a circular air-backed membrane may, for example, have the dimensions of approximately 0.6 to 1.0 mm diameter×0.1 to 0.20 mm thickness.

The membrane of the invention is made of a biocompatible material such that the membrane is capable of reflecting ultrasound waves. The membrane substantially reflects ultrasound waves—specifically, it may reflect greater than 90% of the ultrasound waves. The membrane may reflect up to or greater than 98% of the ultrasound waves. The reflective capacity of the membrane is one indicator of the quality of the membrane as a marker.

As discussed, a device comprising an ultrasound marker according to the present invention can be readily detected by ultrasound within a patient's body or body cavity during an invasive medical procedure. For example, the invention can be used during a biopsy to guide the surgeon or another medical practitioner in the placement of medical instrumentation or apparatus such as biopsy needles, sensors, implants, or other items within the patient's body or body cavity in real time. The invention is also useful for improving visualization of catheters or catheter tips during transcatheter procedures such as tissue ablation for treatment of arrhythmia, transceptal puncture, implantation of sensors, and clipping of leaky mitral heart valves. Other uses of the invention will be apparent from the discussion below.

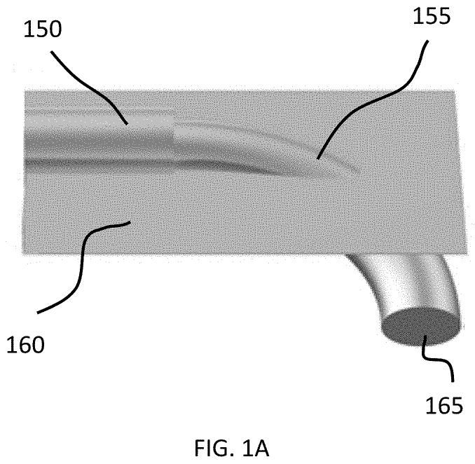

FIG. 1A illustrated one issue with ultrasound imaging of catheters, i.e., the problem of “false tips”. When using ultrasound as the imaging modality during transcatheter procedures, identifying the actual placement of a catheter tip is frequently problematic. The ultrasound “cuts” through the three-dimensional space inside the patient's body in a single plane. Consequently, the ultrasound image will often show a false tip where the ultrasound plane cuts through the catheter instead of displaying the location of the real tip. This inadequacy can be seen in FIG. 1, where a catheter 150 having a tip is imaged via the ultrasound plane 160. The imaging system displays a false tip 155 which is where the ultrasound plane intersects the end of the catheter, whereas the true tip 165 is located below the ultrasound plane and is not visible on a display screen during the medical procedure. It is impossible to know whether the tip of the catheter (or other medical device) on the display screen is the true tip or a false tip via conventional ultrasound techniques, as shown in FIG. 1B. Other medical devices besides catheters also have the same problem with display of false tips.

FIG. 1B shows an ultrasound image of the left atrium of the heart of an implantation in a porcine model using a conventional catheter tip. The locations of the catheter and the catheter tip in the ultrasound image are marked using arrows. Because of the inadequacies of ultrasound imaging as discussed above, it is impossible to know whether the catheter tip in FIG. 1B is a true tip or a false tip.

As can be expected, inaccuracies in displaying the true location of the catheter tip on a display screen can result in imperfect positioning, deployment, or implantation of a surgical, diagnostic, or therapeutic object such as a valve, sensor, probe, stent, or other such medical object in the body. The marker of the present invention is intended to overcome such issues by causing the device having the marker to reflect sound waves such that the position and location of the medical device is accurately and correctly visualized using conventional ultrasound techniques.

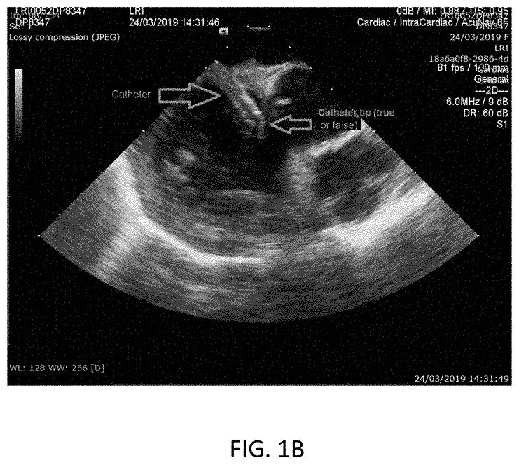

FIGS. 2A and 2B show the marker 102 of the invention which incorporates a membrane 104 that reflects ultrasound waves. In embodiments, the membrane 104 may be a thin air-backed membrane. Air-backed membranes may be made of any material, because air-backing reflects substantially all of ultrasound waves with any material (preferably between about 90% and 99%). (The term “about” as used herein is understood to mean an amount within 1%-5% of the recited value). The marker 102 provides superior positional feedback to a needle or device. It does so by being calibrated to have a specific resonance frequency which differs from that of other objects in a body.

The resonance frequency of the ultrasound marker is controlled by various attributes of the membrane 104 including its size, thickness and composition. Modification of the materials, membrane area 106, and membrane thickness 108 will alter the resonance frequency of the membrane 104 to be suitable for different purposes and/or circumstances. Embodiments of rectangular membranes may have a width and length of approximately 0.5 mm to 2.00 mm and a thickness of 0.05 mm to 0.8 mm. An exemplary rectangular membrane width and length may range from approximately 0.9 mm to 1.34 mm. The membrane thickness 108 may range from 0.1 mm to 0.45 mm. Modifying the physical characteristics of the membrane enables the manufacturer to choose the resonance frequency. One skilled in the art readily recognizes the use of suitable biocompatible materials are included within the scope of the invention where applicable. Non-limiting exemplary materials for the marker are a metal, a metal alloy, titanium, platinum, stainless steel, a shape memory alloy such as but not limited to NITINOL®, silicon, glass, quartz, a ceramic material, a composite material, a metallic or non-metallic nitride, boron nitride, a carbide, a metal oxide, a non-metallic oxide, a polymer based material, and combinations thereof.

When the system is used with color Doppler imaging, the resonance frequency of the membrane is preferably in the range of frequencies made by traditional color Doppler ultrasound transducers. While performing an ultrasound examination, in addition to the standard ultrasound imaging transducer (a first transducer), the practitioner may also use a second transducer calibrated to the resonance frequency of the membrane 104. This second transducer creates sound waves of the resonance frequency of the membrane. The second transducer may be placed 20-25 centimeters from the site of the medical device, which will cause the membrane 104 to vibrate at the resonance frequency, which, in turn, will cause the marker to appear in the color corresponding to its resonance frequency on the ultrasound images.

In color Doppler ultrasound imagers, the resonance frequency of the membrane 104 may be calibrated to be greater than the Doppler shift caused by blood flow. This difference in frequencies will cause the membrane 104 to vibrate at a greater frequency resulting in the membrane 104 displaying a different color than the rest of the image. The unique reverberation period of the membrane will provide a specific comet tail length because after a pulse is sent by the transducer, the reverberation results in a longer interpreted time of flight, which results in a spread of the marker image along the direction of transducer's axis. In one embodiment, a filter can be added to the ultrasound image which removes the color response of blood flow, thereby enhancing the image clarity. Furthermore, the power of the ultrasound transmitter can be adjusted to change how prominently the marker 102 appears on the ultrasound image. In still other embodiments, markers 102 with different resonance frequencies and different color signatures may be added to different points of an object under imaging. Then, by changing the frequency created by the transducer, one can control the part of the object being emphasized on the ultrasound image.

When used with harmonic imaging, the value of the resonance frequency of the membrane is designed to be similar to the value of the ultrasound imager frequencies. Harmonic imagers transmit at the frequency f, and receive at the frequency 2f. Such transducers create sound waves of the resonance frequency of the membrane causing the membrane 104 to vibrate at that frequency. The marker 102 will vibrate at the same frequency more strongly than the surrounding areas and will appear more prominently in the ultrasound image. The amplitude of the signal produced by the marker at the excitation frequency will be a product of the non-linearity of the membrane.

When used with ARFI imaging, the value of the resonance frequency of the membrane is designed to be similar to the value of the ARFI imager frequencies. ARFI imagers transmit at the frequency f. In similar fashion to the use of the membrane with harmonic imagers, such transducers create ultrasound waves of the resonance frequency of the membrane causing the membrane 104 to vibrate at that frequency. The marker 102 will vibrate at the same frequency more strongly than the surrounding areas and will appear more prominently in the ultrasound image.

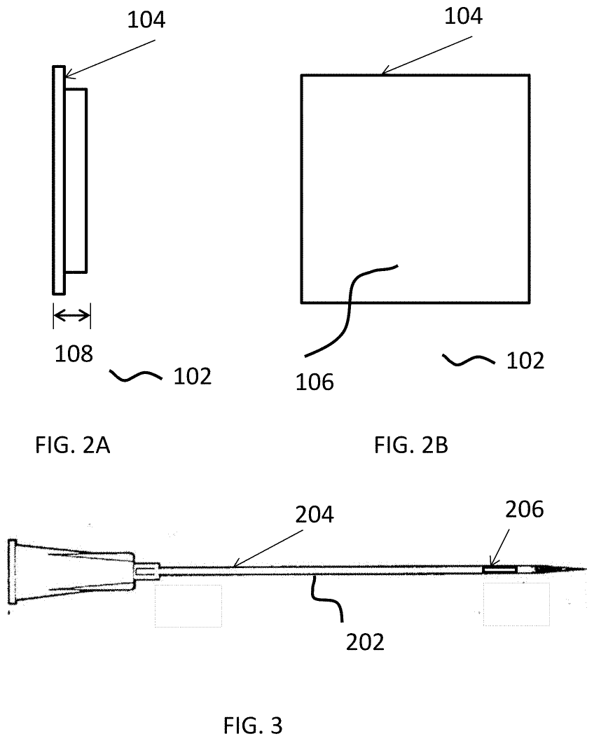

FIG. 3 shows an exemplary embodiment of a needle with a marker attached to said needle 202. The marker 206 is attached to a needle 204 by one of a number of possible methods including welding or embedding, for example. Embedding the marker may be preferable in certain embodiments in that embedding will help maintain the smooth edges of the needle 204.

The invention further comprises a method of detecting a needle having a marker in a patient using an ultrasound transducer. In one example, a needle with a marker attached 202 can be used during ultrasonic guidance of needles while taking a biopsy or in other surgical procedures. A similar apparatus having a marker integrated onto a catheter may be used during angioplasty or other procedures using ultrasonic guidance. Similar apparatuses can also be used, for example, to aid guidance during: implantation of catheter deployed aortic and mitral valves; transeptal needle guidance for crossing into the left atrium; and/or ablation catheter guidance for electrophysiology.

Specifically, one such method comprises (a) inserting the needle having a marker 202 into a target; (b) positioning a first ultrasound transducer over the area of concern in accordance with standard ultrasonic guidance; (c) activating the marker either by activating a second transducer for color Doppler implementations or using the first transducer for harmonic implementations which in turn causes the marker to vibrate which is detectible; and (d) observing the marker on an ultrasound image. This method may be used with other devices having a marker. When viewing the needle having a marker 202 or other device on the ultrasound image, the marker will be easily visible, making navigation easier.

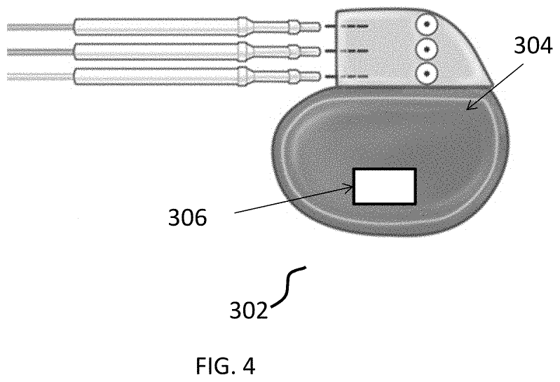

Similarly, the method may also relate to detection of implanted devices having a marker integrated therein or thereon. In particular, FIG. 4 shows an exemplary embodiment of a pacemaker 302 with a marker attached. The marker 306 is integrated onto a pacemaker 304 by one of a number of possible methods including, for example, welding, gluing, integrally manufacturing, or embedding during manufacture. Embedding during manufacture may be preferable for certain devices to help maintain the shape and form of the device 304. The particular location of the marker on the pacemaker may vary.

The pacemaker 302 having a marker may be detected using ultrasound. To use the marker 306, the procedure is the same as described above for use with a marker embedded on a needle 202. Using the procedure with pacemaker 302 having a marker enables a user to easily find the implanted device when performing an ultrasound, making examinations non-invasive and efficient, particularly benefiting the patient when tracking an implant over a long period of time.

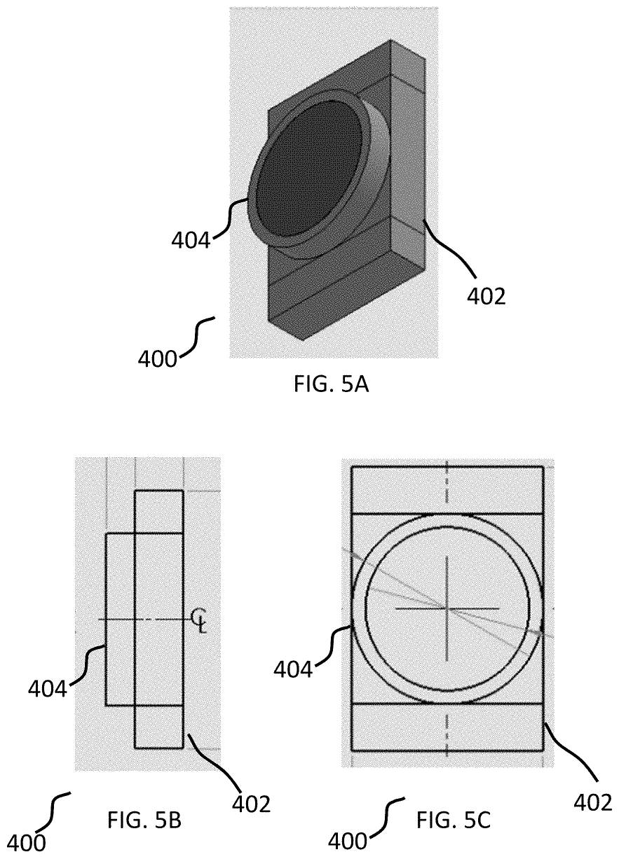

FIG. 5A shows an isometric view of a marker 400 having an air-backed membrane 404 on a housing 402. FIG. 5B shows a side view of the embodiment of the marker 400 shown in FIG. 5A. FIG. 5C shows a top view of the embodiment of the marker 400 shown in FIG. 5A. The housing 402 forms a rectangular solid. It can range in size from about 100 micrometers squared to 250000 micrometers squared. The air-backed membrane 404 is a cylinder which rests on the housing 402. The air-backed membrane 404 may be centered on the housing 402.

The marker of the present invention can be used for ultrasound imaging during any type of invasive medical procedure. In one embodiment, the marker can be used with a vibrating medical device such as a vibrating needle for improved visualization of the needle under ultrasound conditions. In another embodiment, the marker of the present invention can be used during 3D medical holography to assist the surgeon or doctor in visualizing the true position of a medical device in the body during an ultrasound-guided medical or surgical procedure such as (but not limited to) biopsy or implantation. Other uses of the inventive marker will be evident to those of ordinary skill in the art.

It will be appreciated by persons having ordinary skill in the art that many variations, additions, modifications, and other applications may be made to what has been particularly shown and described herein by way of embodiments, without departing from the spirit or scope of the invention. Therefore it is intended that scope of the invention, as defined by the claims below, includes all foreseeable variations, additions, modifications or applications.

What is claimed is:

1. A marker for use with ultrasound imaging of the position of a medical device comprising:
a membrane reflective to ultrasonic waves,
wherein said membrane comprises a membrane area and a membrane thickness, and said membrane vibrates at a resonance frequency.

2. The marker of claim 1, wherein said marker comprises an air-backed membrane.

3. The marker of claim 1, wherein said membrane is mounted on a housing.

4. The marker of claim 1, wherein said membrane comprises silicon.

5. The marker of claim 1, wherein said membrane comprises titanium.

6. The marker of claim 1, wherein said marker is integrated into a needle.

7. The marker of claim 6, wherein said marker is integrated into a biopsy needle.

8. The marker of claim 1, wherein said resonance frequency corresponds to frequencies used in harmonics-type ultrasound imaging.

9. The marker of claim 1, wherein said resonance frequency comprises a range accessible to color Doppler ultrasound imagers.

10. The marker of claim 1, wherein said resonance frequency comprises a range accessible to acoustic radiation force impulse imaging.

11. The marker of claim 1, wherein said marker is integrated into an angioplasty catheter.

12. The marker of claim 1, wherein said resonance frequency is larger than that of a Doppler shift created by blood flow.

13. The marker of claim 1, wherein said membrane has a thickness to diameter ratio between 1:90 and 1:3.

14. The marker of claim 13, wherein said ratio is between 1:6 and 1:4.

15. The marker of claim 1, wherein the membrane is circular and has a diameter between 0.3 to 2 millimeters.

16. The marker of claim 1, wherein the marker is rectangular and has a length and width between 0.5 and 2 millimeters.

17. The marker of claim 1, wherein the marker has a thickness between 0.1 and 0.20 millimeters.

18. The marker of claim 1, wherein the marker has a thickness between 0.05 and 0.8 millimeters.

19. A system comprising:
a medical device having an ultrasound marker mounted on said medical device, said marker comprising a membrane reflective to ultrasonic waves, wherein said membrane comprises a membrane area and a membrane thickness, and said membrane vibrates at a resonance frequency; and
an ultrasound transducer;
wherein said ultrasound transducer transmits at a frequency range corresponding to said resonance frequency.

20. The system of claim 19, wherein said marker comprises an air-backed membrane.

21. The system of claim 19, wherein said membrane comprises silicon.

22. The system of claim 19, wherein said membrane comprises titanium.

23. The system of claim 19, wherein said medical device comprises a needle.

24. The system of claim 23, wherein said needle comprises a biopsy needle.

25. The system of claim 19, wherein said resonance frequency corresponds to frequencies used in harmonics-type ultrasound imaging.

26. The system of claim 19, wherein said resonance frequency comprises a range accessible to color Doppler ultrasound imagers.

27. The system of claim 19, wherein said resonance frequency comprises a range accessible to acoustic radiation force impulse imaging.

28. The system of claim 19, wherein said medical device comprises an angioplasty catheter.

29. The system of claim 19, wherein said resonance frequency is larger than a Doppler shift frequency created by blood flow.

30. The system of claim 19, further comprising a second ultrasound marker comprising a second membrane that is completely reflective to ultrasonic waves, wherein said second membrane comprises a second membrane area and a second membrane thickness such that said second membrane vibrates at a second resonance frequency.

31. A method for detecting a medical device in a body cavity comprising:
a) introducing said medical device having a detectible marker, said marker comprising a membrane reflective to ultrasonic waves into a body cavity, wherein said membrane comprises a membrane area and a membrane thickness, and said membrane vibrates at a resonance frequency;
b) placing an ultrasound transducer over an outside surface of said body cavity in which said ultrasound marker is located, wherein said ultrasound transducer transmits ultrasound waves at a frequency range corresponding to said resonance frequency; and
c) detecting said medical device by reflection of said ultrasound waves sent from said transducer.

32. The method of claim 31, wherein said marker comprises an air-backed membrane.

33. The method of claim 32, wherein said air-backed membrane is mounted on a housing.

34. The method of claim 31, wherein said membrane comprises silicon.

35. The method of claim 31, wherein said membrane comprises titanium.

36. The method of claim 31, wherein said medical device comprises a needle.

37. The method of claim 31, wherein said medical device comprises a heart valve.

38. The method of claim 31, wherein said medical device comprises a catheter.

39. The method of claim 31, wherein said resonance frequency corresponds to frequencies in an ultrasound imager for harmonics-type ultrasound imaging.

40. The method of claim 31, wherein said resonance frequency is in a range accessible to color Doppler ultrasound imagers.

41. The method of claim 31, wherein said marker is integrated into a biopsy needle.

42. The method of claim 31, wherein said membrane is integrated into an angioplasty catheter.

43. The method of claim 31, further comprising the steps of:
a) introducing into said body cavity a second ultrasound marker comprising a second membrane reflective to ultrasonic waves, wherein said second membrane comprises a second membrane area and a second membrane thickness, and said second membrane vibrates at a second resonance frequency; and
b) adjusting said ultrasound transducer, wherein said transducer transmits at a frequency corresponding to said second resonance frequency.

44. A method for rendering an invasive medical device detectable in a patient comprising:
a) introducing said medical device into a body cavity of said patient, said medical device having an ultrasound marker comprising a membrane reflective to ultrasonic waves, wherein said membrane comprises a membrane area and a membrane thickness, said membrane vibrates at a resonance frequency;
b) placing an ultrasound transducer over an outer surface of said body cavity in which said medical device is located, wherein said ultrasound transducer transmits at a frequency range corresponding to said resonance frequency; and
c) detecting said medical device by reflection of ultrasound waves from a second transducer.

45. The method of claim 44, wherein said marker comprises an air-backed membrane.

46. The method of claim 44, wherein said marker comprises silicon.

47. The method of claim 44, wherein said marker comprises titanium.

48. The method of claim 44, wherein said marker is integrated into a needle.

49. The method of claim 44, wherein said resonance frequency comprises a range accessible to color Doppler ultrasound imagers.

50. The method of claim 44, wherein said medical device comprises a biopsy needle.

51. The method of claim 44, wherein said medical device comprises an angioplasty catheter.

52. The method of claim 44, wherein said membrane has a resonance frequency larger than a Doppler shift frequency of blood flow.

53. The method of claim 44, further comprising the steps of:
a) introducing into said body cavity a second medical device having a second ultrasound marker, said second marker comprising a second membrane reflective to ultrasonic waves, wherein said second membrane comprises a second membrane area and a second membrane thickness, and said second membrane vibrates at a second resonance frequency; and
b) adjusting said ultrasound transducer, wherein said transducer transmits at a frequency range corresponding to said second resonance frequency.

54. A method for rendering an invasive medical device detectable in a patient comprising:
a) placing an ultrasound transducer over an outer surface of a body cavity of the patient in which said medical device is located, wherein the medical device comprises an ultrasound marker comprising a membrane reflective to ultrasonic waves and vibrating at a resonance frequency, and wherein said ultrasound transducer transmits at a frequency range corresponding to said resonance frequency; and
b) detecting said medical device by reflection of ultrasound waves from said transducer.
